(19) United States
(12) Patent Application Publication
(10) Pub. No.: US 2022/0226407 A1
(43) Pub. Date: Jul. 21, 2022

(54) PHARMACEUTICAL COMPOSITION COMPRISING THE EXTRACT OF CANNABIS SATIVA AS AN EFFECTIVE INGREDIENT FOR PREVENTING OR TREATING OF OBESITY

(71) Applicant: FAMENITY CO., LTD., Uiwang-si (KR)

(72) Inventors: Tae Wan KIM, Gwacheon-si (KR); Tae Joon KIM, Gwacheon-si (KR)

(21) Appl. No.: 17/460,262

(22) Filed: Aug. 29, 2021

(30) Foreign Application Priority Data
Jan. 18, 2021 (KR) 10-2021-0006906

(51) Int. Cl. A61K 36/185; A61K 31/05; A61K 31/015; A61P 3/04; A23L 33/105; A23L 33/00

(52) U.S. Cl. CPC A61K 36/185; A61K 31/05; A61K 31/015; A23V 2002/00; A23L 33/105; A23L 33/40; A61P 3/04

(57) ABSTRACT

The present disclosure relates to a composition for preventing and treating obesity containing a Cannabis sativa extract, and may provide a composition for preventing and treating obesity, which contains an extract of the natural product Cannabis sativa, and thus has little or no side effects when taken or administered, and may suppress the body weight and body fat gain caused by a high-fat diet in a diet-induced obesity mouse model.

### CROSS-REFERENCE TO RELATED APPLICATION

This application claims priority from and the benefit of Korean Patent Application No. 10-2021-0006906 filed on Jan. 18, 2021, which is hereby incorporated by reference for all purposes as if set forth herein.

### BACKGROUND

### Field

The present disclosure relates to a composition for preventing and treating obesity containing a Cannabis sativa extract. More specifically, the present disclosure provides a composition for preventing and treating obesity, which contains an extract of the natural product Cannabis sativa, and thus has little or no side effects when taken or administered, and may suppress body weight and body fat gain caused by a high-fat diet in a diet-induced obesity mouse model.

### Discussion of the Background

In recent years, in Korea, the intake of fat from food has increased due to economic growth and westernization of diet, and metabolic diseases such as obesity, diabetes, hyperlipidemia, hypertension, arteriosclerosis and fatty liver have tended to increase due to lack of exercise. In addition, obesity is unattractive for young people who prefer slim bodies, and continued obesity leads to various diseases which need to be treated.

Obesity refers to a phenomenon in which excess calories are accumulated as fats in the body by ingesting more calories than the calories consumed. The World Health Organization (WHO) reported that about 400 million people in the world over the age of 15 are classified as obese, and 1.6 billion people are overweight (September 2006). In line with this trend, obesity has emerged as a serious social problem, not just an individual disease. Obesity is known to be caused by genetic factors, environmental factors resulting from westernized dietary habits, and psychological factors resulting from stress, but the exact cause or mechanism of obesity has not been clearly identified.

The goals of obesity treatment are broadly divided into two. The first goal is to lose weight by burning excess fat, and the second goal is to improve metabolic imbalance. Abdominal obesity is closely related to symptoms such as insulin resistance, type 2 diabetes, hypertension and lipid metabolism abnormalities, and acts as a strong risk factor for early arteriosclerosis, ischemic heart disease and cerebrovascular disease. Thus, currently, the treatment of obesity aims not only at losing weight, but also at ameliorating metabolic abnormalities by early eliminating factors that cause cardiovascular disease.

Therapeutic drugs that are currently used to treat obesity can be broadly divided into drugs, which affect appetite by acting on the central nervous system, and drugs which suppress uptake by acting on the gastrointestinal tract. Commercially available drugs that act on the central nervous system according to the respective mechanisms include drugs such as fenfluramine and dexfenfluramine, which inhibit the serotonin (5-HT) nervous system, drugs such as ephedrine and caffeine, which act on the noradrenaline nervous system, and recently commercially available drugs such as sibutramine, which inhibit obesity by simultaneously acting on the serotonin and noradrenaline nervous system. In addition, typical drugs that inhibit obesity by acting on the gastrointestinal tract include orlistat approved for use as an obesity therapeutic agent that inhibits fat absorption by inhibiting lipase in the gastrointestinal tract.

However, among the conventional drugs that have been used, drugs such as fenfluramine cause side effects such as primary pulmonary hypertension or heart valve disease, and thus the use thereof was recently prohibited. Other drugs cause problems such as reduced blood pressure or lactic acidosis, and thus are inapplicable to patients with heart failure or renal disease.

First of all, in order to find a more improved method for treating obesity, there has been recent interest in the mechanism of regulating energy metabolism. Under the premise that the compounds of this family should have higher safety (lower toxicity), studies have been conducted on proteins that influence fat accumulation and signals that are accumulated as fat when humans take a high-fat diet. Through studies on signals for inhibiting the expression of these fat-accumulating proteins and decomposing already accumulated fat and studies on proteins that are involved therein, peptides that promote lipolysis have been developed.

Meanwhile, studies focused on suppressing obesity by controlling diet intake and energy consumption have also been actively conducted. Hypothalamus and motor nervous, autonomic nervous and peripheral nervous systems are all involved in the regulation of food intake behaviors, and the central nervous system, especially the hypothalamus, plays an important role in the etiology of obesity. Typical factors that are secreted from the hypothalamus include neuropeptide Y, POMC/CART, melanocortin receptor, norepinephrine, serotonin, and the like. Current strategies for developing obesity therapeutic agents include reducing food intake, inhibiting calorie absorption, promoting exothermic reactions, regulating energy metabolism, and regulating signal transduction through the nervous system.

Representative drugs for reducing weight gain caused by obesity include sibutramine, which is a drug that suppresses appetite, and orlistat that reduces the uptake of fat contained in food by inhibiting lipase in the body. However, in the case of sibutramine, side effects such as increased blood pressure, insomnia, dry mouth, and dizziness are relatively common. In addition, sibutramine has a disadvantage in that it is inapplicable to patients with cardiovascular disease or uncontrolled hypertension. In addition, in the case of orlistat, side effects such as diarrhea, fat stool, and fecal incontinence are common, and the effect of the drug orlistat is not remarkable for Koreans who have less fat intake than Westerners.

Therefore, there is a need for constant studies on drugs having an excellent anti-obesity effect while being safer.

### PRIOR ART DOCUMENTS

### Patent Documents

(Patent Document 1) KR 10-2020-0180279 B1

### SUMMARY

An object of the present disclosure is to provide a composition for preventing and treating obesity, which contains an extract of the natural product Cannabis sativa, and thus has little or no side effects when taken or administered.

Another object of the present disclosure is to provide a composition for preventing and treating obesity, which may suppress body weight and body fat gain caused by a high-fat diet in a diet-induced obesity mouse model.

To achieve the above objects, a composition for preventing and treating obesity according to one embodiment of the present disclosure contains a Cannabis sativa extract as an active ingredient.

The Cannabis sativa extract contains cannabidiol and terpene.

The composition suppresses obesity by inhibiting adipocyte differentiation-stimulating factors.

The adipocyte differentiation-stimulating factors include PPARγ (peroxisome proliferator-activated receptor γ), C/EBPα (CCAAT/enhancer-binding protein α), and fatty acid synthase.

The Cannabis sativa extract is obtained by extraction with an extraction solvent selected from the group consisting of water, a C₁ to C₆ lower alcohol, and a mixture thereof.

A food composition for preventing obesity according to another embodiment is produced to contain the above-described composition.

A pharmaceutical composition for treating obesity according to still another embodiment is produced to contain the above-described composition.

Hereinafter, the present disclosure will be described in more detail.

As used herein, the term “preventing” refers to any action of suppressing or delaying obesity or complications thereof by administering the extract or the composition containing the same. It will be obvious to those skilled in the art that, when the extract of the present disclosure, which has a therapeutic effect on obesity or complications thereof, is administered in an early stage of obesity or complications thereof or before the onset thereof, it may prevent this obesity or complications thereof.

As used herein, the term “treating” refers to any action of alleviating or beneficially changing symptoms of obesity or complications thereof, for example, hyperlipidemia or other diseases caused thereby, by administering the extract or the composition containing the same. With reference to the data presented by the Korean Medical Association, etc., those of ordinary skill in the art to which the present disclosure pertains may understood exact criteria for obesity or complications thereof against which the composition of the present disclosure is effective, and may determine the degree of amelioration, improvement or treatment of obesity or complications thereof.

As used herein, the term “obesity” does not simply refer to having a lot of weight, but refers to a condition in which body fat is excessively accumulated. That is, a person who has a high body fat percentage even though appearing to have a normal body weight may be considered obese. Usually, obesity is determined using body mass index (BMI), and BMI criteria are as follows: BMI of 23 to 24.9: overweight; BMI of 25 to 29.9: mild obesity; BMI of 30 to 34.9: moderate obesity; and BMI of 35 or more: severe obesity.

As used herein, the term “body fat” refers to adipose tissue constituting the body. Body fat is widely distributed around subcutaneous tissues, mammary glands and kidneys, and stored fat functions to protect the internal organs and regulate the body temperature in addition to being used for energy generation. A condition in which stored fat is excessively accumulated is referred to as obesity. In obesity, the amount of body fat is more important than body weight from the viewpoint of preventing complications. It is known that the accumulation of visceral fat in the abdominal cavity is more deeply related to glucose and lipid metabolism abnormalities, hypertension and coronary artery diseases than the accumulation of subcutaneous fat. In the present disclosure, the term “body fat” includes both subcutaneous fat and visceral fat.

As used herein, the term “extract” not only means a crude extract that is commonly used in the art as described above, but also includes, in a broad sense, a fraction obtained by fractionating the extract. That is, the term “extract” includes not only an extract obtained using the above-described extraction solvent, but also one obtained by additionally applying a purification process to the extract. For example, the term “extract” as used herein also include a fraction obtained by passing the extract through an ultrafiltration membrane having a certain molecular weight cut-off value, and fractions obtained by additionally performing various purification processes, such as separation by various chromatography systems (manufactured for separation according to size, charge, hydrophobicity or affinity).

A composition for preventing and treating obesity according to one embodiment of the present disclosure contains a Cannabis sativa extract as an active ingredient.

Cannabis sativa is an annual plant belonging to the genus Cannabis of the family Cannabaceae, and is flowing plant species including three different subspecies: C. sativa, C. indica, and C. ruderalis.

As far as is known, about 400 compounds have been found in Cannabis sativa, and most of them are cannabinoids, terpenes, and phenolic compounds. Among them, cannabinoids are known as representative active ingredients of Cannabis sativa. About 90 kinds of cannabinoids have been identified to date, and a number of ingredients found only in Cannabis sativa are also known. Cannabinol (CBN) was isolated from Cannabis sativa in 1899, but it was later found that the cannabinol was not a single compound. Since cannabidiol (CBD) and tetrahydrocannabinol (THC), which are pure compounds, were isolated from Cannabis sativa in the 1930s, studies on the components of Cannabis sativa have been more actively conducted.

Efforts to develop drugs using specific components of Cannabis sativa have also been continued, and among these specific components, THC and CBD, which are major compounds of Cannabis sativa, have attracted the most attention for therapeutic purposes. Some studies indicated that CBD has no phrenotropic action and is effective in reducing pain and controlling epileptic seizures.

In addition, more than 100 terpene-based compounds that play a role in the flavor and taste of Cannabis sativa were also found in Cannabis sativa, and are present as various monoterpenoids and sesquiterpenoids. Terpenes have been found to be related to various pharmacological actions such as anti-inflammatory action, but studies on terpene compounds extracted from Cannabis sativa are still insufficient compared to THC.

The Cannabis sativa extract contains cannabidiol and terpene.

Cannabidiol (CBD) is one of the main components of Cannabis and is a compound that is much comparable with tetrahydrocannabinol (THC).

In the case of Korea, cannabidiol has been designated as a narcotic, and thus many studies thereon have not been conducted, but in foreign countries, cannabidiol has been actually used as a medical drug for relieving symptoms such as pain, memory disorder, and anxiety, and active studies thereon have been conducted.

Tetrahydrocannabinol (THC) is a major psychotropic component of the Cannabis sativa plant, and THC is psychotropic only in a decarboxylated state. THC has a structure similar to that of CBD, but it is known that THC induces excitement and has an apoptotic effect in some cancers, whereas CBD has been less studied compared to THC and does not induce excitement.

The most well-studied cannabinoids include tetrahydrocannabinol (THC), cannabidiol (CBD) and cannabinol (CBN). Other cannabinoids include, for example, cannabichromene (CBC), cannabigerol (CBG), cannabinidiol (CBND), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM).

Terpenes are known to exhibit better effects when acting together with cannabinoids such as CBD and THC, and may improve the uptake of cannabinoids, overcome the bacterial defense mechanism and minimize side effects.

Cannabis sativa has been used in various ways in the past depending on parts thereof. Specifically, it is known that the leaf of Cannabis sativa has the effect of killing roundworms, and that, when hair is washed with water obtained by boiling the leaf of Cannabis sativa, the hair grows long and becomes abundant. In addition, the leaf of Cannabis sativa was used against asthma or old cough or roundworms, or as an analgesic, anesthetic or diuretic agent. There is a record that the root of Cannabis sativa was used for the treatment of difficult delivery and “placenta not coming out”, the removal of extravasated blood, and the treatment of urolithiasis, and was taken as a water decoction. There is a record that the shell of Cannabis sativa was used for the treatment of bruises and fever-type intestinal pain, and the flower of Cannabis sativa was used for paralysis symptoms and itching. The flower spike of Cannabis sativa was used for difficulty delivery, constipation, gout, manic depressive psychosis, insomnia, and the like. In particular, the seeds of Cannabis sativa are rich in L-arginine, and thus may exhibit a tension relieving effect by releasing muscle tension, and cannabinoids, which are the unique components of Cannabis sativa, are effective in pain relief and tension relief.

The composition suppresses obesity by inhibiting adipocyte differentiation-stimulating factors, in which the adipocyte differentiation-stimulating factors include PPARγ (peroxisome proliferator-activated receptor γ), C/EBPα (CCAAT/enhancer-binding protein α), and fatty acid synthase.

The composition for preventing and treating obesity containing a Cannabis sativa extract as an active ingredient according to the present disclosure may suppress obesity by inhibiting adipocyte differentiation-stimulating factors.

In the present disclosure, the adipocyte differentiation-stimulating factors include PPARγ (peroxisome proliferator-activated receptor γ), C/EBPα (CCAAT/enhancer-binding protein α), and fatty acid synthase, but they are not particularly limited as long as they do not impair the effect of the present disclosure.

The adipocyte differentiation-stimulating factors are keys among transcriptional regulatory factors, and serve to promote the expression of adipogenesis-related genes and differentiation into adipocytes through the interaction between PPARγ and C/EBPα. Thus, fat accumulation may be inhibited by inhibiting the adipocyte differentiation-stimulating factors.

Obesity refers to a condition in which adipocytes proliferate and differentiate in the body due to metabolic disorders, and hence fat is excessively accumulated in the body. Obesity may cause related complications including metabolic syndrome accompanied by hypertension, diabetes and dyslipidemia.

The composition for preventing and treating obesity containing a Cannabis sativa extract as an active ingredient according to the present disclosure has an excellent effect of preventing a metabolic disease selected from the group consisting of hyperlipidemia, fatty liver, cardiovascular disease, and arteriosclerosis.

The term “metabolic disease” refers to a condition or disease that is closely related to obesity or is caused by obesity. Specifically, the metabolic disease may be one or more selected from the group consisting of hyperlipidemia, fatty liver, cardiovascular disease, and arteriosclerosis.

The hyperlipidemia refers to a case in which the concentrations of lipid components (especially cholesterol and triglyceride) in the blood are high. Generally, a blood cholesterol concentration higher than 240 mg/dl or a blood triglyceride concentration of 200 mg/dl or higher is referred to as hyperlipidemia. Hyperlipidemia may be caused by a genetic predisposition, obesity, dietary habits, diabetes, nephrotic syndrome, or hypothyroidism.

The composition for preventing and treating obesity according to the present disclosure may be applied in a broad sense to all conditions in which it is required to lower lipid concentrations in blood.

The fatty liver refers to a condition or disease in which fat is excessively accumulated in liver cells due to a hepatic fat metabolism disorder.

The arteriosclerosis refers to a condition or disease in which blood circulation to organs and tissues in the body is lowered due to the thickening and decreased elasticity of the arterial wall. In addition, the arteriosclerosis is meant to include “atherosclerosis” which means a condition or disease in which blood circulation is lowered by narrowing of the lumen due to plaques formed by deposition of other substances such as fat and cholesterol on the inner wall of the artery. Arteriosclerosis may occur anywhere in the body. If arteriosclerosis occurs in the blood vessels in the heart, it may cause coronary artery diseases such as angina pectoris and myocardial infarction, and if arteriosclerosis occurs in the brain, it may cause cerebral infarction, and if arteriosclerosis occurs in the kidney, it may cause kidney failure and the like.

Specifically, the composition for preventing and treating obesity containing a Cannabis sativa extract as an active ingredient according to the present disclosure may ameliorate, prevent and treat the above-described obesity or metabolic disease. In addition, the composition may suppress the body weight or body fat gain caused by various factors, and more preferably, may prevent obesity or metabolic disease caused by a high-fat diet.

Long-term administration of the high-fat diet may cause body weight and body fat gain, induce adipocyte differentiation and fat accumulation over time, increase body weight and the content of body fat, and increase not only the concentrations of endotoxins, triglycerides and total cholesterol in serum, but also the expression of adipocyte differentiation-related hormones, thus inducing obesity or metabolic disease.

The composition for preventing and treating obesity containing a Cannabis sativa extract as an active ingredient according to the present disclosure may reduce body weight and body fat gain by inhibiting adipocyte differentiation and fat accumulation, and inhibit the expression of adipocyte differentiation-related hormones

The Cannabis sativa extract is obtained by extraction with an extraction solvent selected from the group consisting of water, a C₁ to C₆ lower alcohol, and a mixture thereof.

Specifically, the Cannabis sativa extract as a natural extract may be obtained by a method including steps of: crushing a natural product to obtain a sample; leaching the sample with an organic solvent; drying the leached sample; re-leaching the dried sample with an organic solvent; drying the re-leached sample; leaching the dried sample with water; and leaching.

The natural extract obtained by extraction with the organic solvent may be further subjected to a fractionation step using an organic solvent.

The extraction solvent may be used in an amount equal to 2 to 50 times, more specifically 2 to 20 times, the weight of the sample. For leaching and extraction, the sample may be left to stand in the extraction solvent for 1 to 72 hours, more specifically 24 to 48 hours.

The extract may be prepared in a powder state by additional processes such as reduced pressure distillation and freeze drying or spray drying, and is obtained by an extraction method selected from the group consisting of a solvent extraction method, an ultrasonic extraction method, a reflux extraction method, a leaching method, a fermentation method, and a processing method.

The ultrasonic extraction method is performed by extraction using water or a 50 to 100% alcohol having 1 to 6 carbon atoms as an extraction solvent at 30 to 50° C. for 0.5 to 2.5 hours. Specifically, the ultrasonic extraction method includes performing extraction using water or a 70 to 80% alcohol having 1 to 6 carbon atoms as an extraction solvent at 40 to 50° C. for 1 to 2.5 hours.

The reflux extraction method is performed by refluxing 10 to 30 g of the crushed natural product in 100 mL of water or a 50 to 100% alcohol having 1 to 6 carbon atoms for 1 to 3 hours. More specifically, the reflux extraction method is performed by refluxing 10 to 20 g of the crushed natural product in 100 mL of water or a 70 to 90% alcohol having 1 to 4 carbon atoms for 1 to 2 hours.

The leaching method is performed by using water or a 50 to 100% alcohol having 1 to 6 carbon atoms as an extraction solvent at 15 to 30° C. for 24 to 72 hours. More specifically, the leaching method is performed by using water or a 70 to 80% alcohol having 1 to 6 carbon atoms as an extraction solvent at 20 to 25° C. for 30 to 54 hours.

After extraction, the extract may be fractionated sequentially using fresh fractionation solvents. The fractionation solvent that is used for fractionation of the extract is any one or more selected from the group consisting of water, hexane, butanol, ethyl acetic acid, ethyl acetate, methylene chloride, and mixtures thereof. Preferably, the fractionation solvent is ethyl acetate or methylene chloride.

Preferably, the composition for preventing and treating obesity containing a Cannabis sativa extract as an active ingredient may additionally contain an extract of Galium verum L. var., an extract of Artemisia sylvatica Maxim. and an extract of Nigella sativa.

The Galium verum L. var. has 8-10 verticillate leaves, which have pointed ends and are each 2 to 3 cm in length and 1.3 to 2 cm in width. The backside of the leaf has many hairs along with nodes and inflorescences. Galium verum L. var. is a perennial plant that is distributed all over Korea and grows mainly on the edges of mountain forests, grasslands and riversides in northern regions of Korea. Galium verum L. var. is distinguished by having short hairs and thorn-shaped hairs on the front sides of the leaves compared to compared to Galium odoratum, and has short hairs and thorn-shaped hairs on the front surfaces of the leaves compared to Galium verum var. trachycarpum.

The Artemisia sylvatica Maxim. is a perennial dicotyledonous plant belonging to the family Asteraceae of the order Campanulales, and grows in mountain forests. Leaves from the roots of Artemisia sylvatica Maxim. remain until flowering, spread in a rose flower shape, have an egg shape or a long oval shape, and have pointed ends. The leaf thereof is 11 to 20 cm in length and 7 to 9.5 cm in width, and the surface thereof has slightly curly hairs. The backside of the leaf has cobweb-like hairs, and the leaf has pointed teeth at the edges thereof. Leaves from the stems are similar to but different in size from the leaves from the roots.

Nigella sativa is an annual or biennial dicotyledonous plant belonging to the family Ranunculaceae of the order Ranunculales. Nigella sativa is an annual plant that grows to a height of about 50 to 80 cm. The leaves thereof are split pinnately 3 to 4 times to form the shape of cosmos leaves and are glossy. The flowers of Nigella sativa bloom in June through July, are light purple in color, and each hang at the end of each branch. The fruits thereof are plump and ball-shaped, and the seeds thereof are small and black and ripen in August through September. Nigella sativa is native to southern Europe. The strawberry-scented seeds of Nigella sativa are widely distributed in India, Egypt, Greece, and Turkey, and have a spicy nutmeg taste. The roasted and dried seeds of Nigella sativa are used to add flavor to curry, vegetable and bean dishes.

When the natural extracts are used in combination, they may exhibit a synergistic effect, and thus exhibit an excellent effect of suppressing body weight and body fat gain caused by a high-fat diet in a diet-induced obesity mouse model, thus exhibiting an excellent effect of preventing and treating obesity.

In addition, as the extract of Galium verum L. var., an extract of Artemisia sylvatica Maxim. and the extract of Nigella sativa are additionally contained, it is possible to provide a composition having excellent palatability by neutralizing the unique taste and flavor of the Cannabis sativa extract.

Preferably, the composition of the present disclosure may contain, based on 100 parts by weight of Cannabis sativa extract, 20 to 40 parts by weight of the extract of Galium verum L. var., 20 to 40 parts by weight of the extract of Artemisia sylvatica Maxim., and 20 to 40 parts by weight of the extract of Nigella sativa.

When the extracts are used in combination in amounts within the above-described ranges, it is possible to provide a composition having excellent palatability while exhibiting an excellent effect of preventing and treating obesity.

The composition for preventing and treating obesity containing a Cannabis sativa extract as an active ingredient according to the present disclosure may be used in various applications.

A food composition for preventing obesity according to another embodiment of the present disclosure is produced to contain the above-described composition.

As used herein, the term “functional food” refers to foods produced and processed using functional raw materials or ingredients beneficial to human health pursuant to Health Functional Foods Act No. 6727, and the term “functionality” means controlling nutrients for the structure or functions of the human body or providing beneficial effects to health purposes, such as physiological effects.

A pharmaceutical composition for treating obesity according to still another embodiment of the present disclosure is produced to contain the above-described composition.

The dosage form of a medicament of the present disclosure may be preferred form selected depending on the method of use thereof, and specific examples of the dosage form include granules, powders, syrups, liquids, suspensions, decoctions, infusions, tablets, suppositories, injections, spirits, capsules, pills, and soft or hard gelatin capsules.

In addition, if necessary, the medicament of the present disclosure may further contain an excipient, a filler, an extender, a binder, a disintegrant, a lubricant, a preservative, an antioxidant, an isotonic agent, a buffer, a film-forming agent, a sweetening agent, a solubilizing agent, a base agent, a dispersing agent, a wetting agent, a suspending agent, a stabilizer, a colorant, a fragrance, etc. which are commonly used in the art.

In the manufacture of the medicament, the content of the composition for preventing and treating obesity according to the present disclosure may vary depending on the form of the medicament, and the dosage thereof may be easily adjusted by those skilled in the art depending on the type of subject to be treated, the route of administration, the subject's weight, sex, age, and the severity of the disease.

### BRIEF DESCRIPTION OF THE DRAWINGS

FIG. 1 shows the results of measuring percent lipid accumulation in differentiated adipocytes, treated with a composition according to one embodiment of the present disclosure, by a spectrophotometer.

FIG. 2 shows the inhibitory effect of a composition according to one embodiment of the present disclosure on adipocyte differentiation-stimulating factors in differentiated adipocytes.

FIG. 3 shows the results of comparing mouse body weight and food intake between mice, to which a high-fat diet was administered and a composition according to one embodiment of the present disclosure was orally administered, and a control group.

### DETAILED DESCRIPTION OF THE ILLUSTRATED EMBODIMENTS

Hereinafter, examples of the present disclosure will be described in detail so that those of ordinary skill in the art can easily carry out the present disclosure. However, the present disclosure may be embodied in a variety of different forms and is not limited to the examples described herein.

### Production Example 1: Production of Extracts

1. Production of Cannabis sativa Extract

Cannabis sativa including leaves and flowers was washed clean with running water, and then completely dried naturally. The dried Cannabis sativa was crushed with a mixer and then prepared into powder. 100 g of Cannabis sativa powder was immersed in 1,000 g of ethanol and eluted at 40° C. for 48 hours. Thereafter, the solid was removed by centrifugation, and the remaining supernatant was collected and filtered. The filtrate was subjected to a conventional concentration process under reduced pressure to obtain a Cannabis sativa extract (CE) containing cannabidiol and terpene at a concentration of 0.15 mg/ml.

2. Production of Other Natural Extracts

First, Galium verum L. var. was washed, dried and then crushed. The crushed Galium verum L. var. was added to a 60% ethanol and extracted for 2 hours. The extract was cooled and then filtered through Whatman filter paper. The filtrate was collected, thus producing a Galium verum L. var. extract (GE).

An Artemisia sylvatica Maxim. extract (AE) and a Nigella sativa extract (NE) were produced according to the same method as the method for producing the Galium verum L. var. extract (GE).

3. Production of Extract mixtures

The Cannabis sativa extract (CE), the Galium verum L. var. extract (GE), the Artemisia sylvatica Maxim. extract (AE) and the Nigella sativa extract (NE) were mixed together as shown in Table 1 below to obtain extract mixtures.

TABLE 1

| | OT1 | OT2 | OT3 | OT4 | OT5 | OT6 |
|---|---|---|---|---|---|---|
| CE | 100 | 100 | 100 | 100 | 100 | 100 |
| VE | — | 10 | 20 | 30 | 40 | 50 |
| RE | — | 10 | 20 | 30 | 40 | 50 |
| NE | — | 10 | 50 | 30 | 40 | 50 |

(unit: parts by weight)

### Test Example 1: Cytotoxicity Test

To test the toxicity of each of the Cannabis sativa extract (CE) (OT1) and the extract mixtures (OT2 to OT6) produced in Production Example 1, differences in toxicity and side effects caused by administration of the extract mixtures in repeated-dose toxicity tests for rats were examined.

6-week-old male and female SD rats were divided into a plurality of groups, each consisting of 10 rats (5 male rats and 5 female rats), and each of the Cannabis sativa extract (CE) (OT1) and the extract mixtures (OT2 to OT6) was administered to the rats. Each of the Cannabis sativa extract and the extract mixtures was dissolved in a 0.5% methylene chloride (MC) solution and then administered orally once at the same time in the morning every day. This administration was repeated for 13 weeks. Each of the extract mixtures was administered once a day at a daily dose of 3.75 mg/kg to 5 mg/kg. Thereafter, mortality, general symptoms, weight changes, and feed and water intakes were observed.

As a result, no death occurred within the test period. In view of the above test results, it was confirmed that the Cannabis sativa extract (CE) (OT1) and the extract mixtures (OT2 to OT6) had no toxicity problem.

### Test Example 2: Inhibitory Effect on Adipocyte Differentiation and Lipid Accumulation

Adipocyte Differentiation

In order to measure the inhibitory effects of the Cannabis sativa extract (CE) (OT1) and extract mixtures (OT2 to OT6), produced in Production Example 1, on adipocyte differentiation and lipid accumulation, 3T3-L1 preadipocytes were cultured in a 12-well plate until confluency was reached. Then, the cells were treated with each of OT1 to OT6, and induced to differentiate in an MDI medium (high-glucose DMEM (Dulbecco's Modified Eagle's Medium) containing 10% FBS (fetal bovine serum), 500 μM methylisobutylxanthine, 1 μM dexamethasone, and 10 μg/mL insulin) which is an adipocyte differentiation-inducing medium.

The medium was replaced with a medium containing 10% FBS DMEM, 500 μM methylisobutylxanthine, 1 μM dexamethasone and 10 μg/mL insulin, and after 3 days, the medium was replaced with a medium containing 10 μg/mL insulin. After 6 days, differentiation was induced for 10 days while the 10% FBS DMEM medium was replaced every two days.

After 10 days, the differentiated cells were fixed in PBS (phosphate buffered saline) containing 4% paraformaldehyde for 1 hour, washed twice with PBS containing 60% isopropanol, stained with Oil-Red-O staining solution for 15 minutes, and then washed with running water. After completion of the staining, the cells were photographed, and the stained portion was quantified using the Image J program.

Inhibitory Effect on Lipid Accumulation

In order to evaluate the inhibitory effect of each of the Cannabis sativa extract and the extract mixtures on lipid accumulation in the differentiated cells, percent lipid accumulation was measured with a spectrophotometer. In the experiment, as controls for OT1 to OT6, cells treated with phloretin and untreated cells were used, respectively.

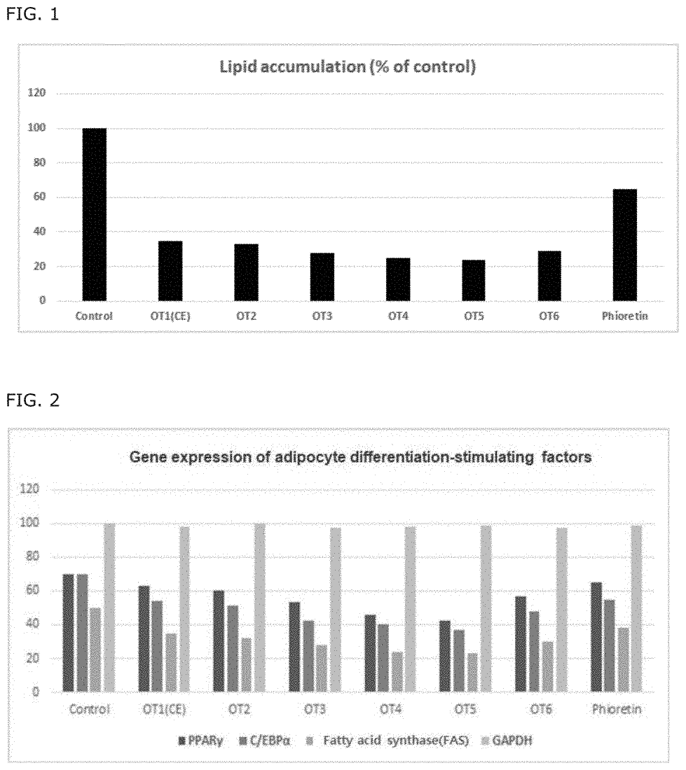

The results of the measurement are shown in FIG. 1.

As shown in FIG. 1, it was confirmed that adipocyte differentiation in the groups treated with each of the Cannabis sativa extract (CE) (OT1) and extract mixtures (OT2 to OT6) of the present disclosure was significantly inhibited compared to that in the control group, and the degree of inhibition of adipocyte differentiation in these groups was also significantly higher than that in the group treated with phloretin.

In addition, as a result of measuring the amount of lipid accumulation in the cells, it was confirmed that lipid accumulation in the groups treated with each of the Cannabis sativa extract (CE) (OT1) and extract mixtures (OT2 to OT6) was significantly inhibited to about 30% of that in the control group, and the degree of inhibition of lipid accumulation in these groups was also two times or higher than that in the group treated with phloretin.

### Test Example 3: Inhibitory Effect on Adipocyte Differentiation-Stimulating Factors

According to the same method as that of Test Example 2, preadipocytes were induced to differentiate. Then, for the eight test groups, including the groups treated with each of the Cannabis sativa extract (CE) (OT1) and extract mixtures (OT2 to OT6) of the present disclosure, the group treated with phloretin, and the untreated group, the preadipocytes induced to differentiate were harvested.

Thereafter, primers for amplification of obesity-related genes, including PPARγ (peroxisome proliferator-activated receptor γ), C/EBPα (CCAAT/enhancer-binding protein α) and fatty acid synthase (FAS) genes, were constructed as shown in Table 2 below, and RT-PCR was performed using the constructed primers. The PCR was performed for 35 cycles, each consisting of denaturation at 95° C. for 1 min, annealing at 54° C. for 1 min, and extension at 72° C. for 1 min.

GAPDH (glyceraldehyde-3-phosphate dehydrogenase) is an enzyme which is involved in glycolysis, an essential metabolic process in cells, and is a gene that is always expressed in cells and whose expression level does not change well. This gene is a good intracellular standard for a target gene to be investigated for changes in its expression level in cells.

TABLE 2

| Primer | Nucleotide sequence |
|---|---|
| GAPDH-F | 5′-CCC TTA TTG ACC TCA ACT ACA TGG T-3 |
| GAPDH-R | 5′-GAG GGG CCA TCC ACA GTC TTC TG-3′ |
| PPARγ2-F | 5′-GTT TTA TGC TGT TAT GGG TG-3′ |
| PPARγ2-R | 5′-GTA ATT TCT TGT GAA GTG CT-3′ |
| C/EBPα-F | 5-TTA CAA CAG GCC AGG TTT CC-3′ |
| C/EBPα-R | 5′-GGC TGG CGA CAT ACA GTA CA-3′ |
| FAS-F | 5′-TTG CTG GCA CTA CAG AAT GC-3 |
| FAS-R | 5-AAC AGC CTC AGA GCG ACA AT-3′ |

After performing the RT-PCR, the expressed portion was quantified using the Image J program, and the results are shown in FIG. 2.

As shown in FIG. 2, it was confirmed that, in the test groups treated with the Cannabis sativa extract (CE) (OT1) and extract mixtures (OT2 to OT6) of the present disclosure, respectively, the expression of PPARγ (peroxisome proliferator-activated receptor γ), C/EBPα (CCAAT/enhancer-binding protein α) and fatty acid synthase (FAS) genes, which play a pivotal role in stimulating adipocyte differentiation, was significantly inhibited compared to that in the control groups, and particularly, the Cannabis sativa extract and the extract mixtures also had significantly better effects than phloretin.

### Test Example 4: Effect of Reducing Body Weight Gain Caused by High-Fat Diet Therapy

High-Fat-Diet Therapy Test Groups

Each of the Cannabis sativa extract (CE) (OT1) and extract mixtures (OT2 to OT6) of the present disclosure and phloretin was dissolved in 0.5% DMSO, and then was administered orally to each mouse of each test group at a dose of 20 mg/kg every other day for 14 weeks. Another mouse group was administered DMSO in the same manner.

The body weight of each mouse was measured in units of 0.01 g at the same time every day, and the food intake was measured once a week (every 7 days).

After 12 weeks of administration of the high-fat diet, adipose tissue was isolated from each mouse and weighed.

Standard Diet Therapy Test Group

The remaining one mouse group was administered DMSO in the same manner using standard diet therapy at the same temperature under the same environmental conditions as the above-described high-fat diet therapy, and then the body weight of each mouse was measured. After 12 weeks of administration of the diet, adipose tissue of each mouse was isolated, and the size and weight thereof were measured and used as a negative control.

Isolation of Adipose Tissue

The adipose tissues isolated from the high-fat-diet therapy test groups and the standard diet therapy test group were subjected to histological examination using a hematoxylin and eosin (H&E) staining method.

Specifically, each adipose tissue was embedded in paraffin, frozen, sectioned to a thickness of 8 μm using a cryocut microtome, and then mounted on a slide glass. Each of the slides having the section mounted thereon was deparaffinized by 5 minutes of immersion in xylene, and hydrated using ethanol at gradually decreasing concentrations (100%-95%-85%-70% for 2 minutes each).

Thereafter, each slide was washed with water to remove the remaining ethanol, and stained with hematoxylin for 6 minutes. Then, each slide was immersed in and taken out of a mixed solution of 1% hydrochloride-ethanol (HCl-EtOH), and this process was repeated three times so that the hematoxylin was sufficiently absorbed into the tissue. Then, the slide was immersed in and taken out of 0.5% ammonia water, and this process was repeated 10 times, thereby fixing the stain.

The tissue section stained with hematoxylin was stained again with eosin for 1 minute and dehydrated using ethanol at increasing concentrations (70%-85%-95%-100% for 2 minutes each).

Effect on Weight Loss

Each of the dehydrated tissue slides was washed clean by 5 minutes of immersion in xylene, and then completely dried at room temperature. Then the section of the tissue was observed under a microscope, and the body weight was measured.

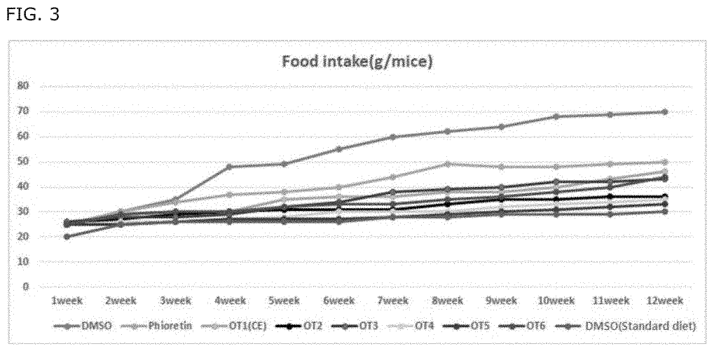

The results of the measurement are shown in FIG. 3.

As shown in FIG. 3, it was confirmed that, among the mouse groups to which the high-fat-diet therapy was applied, the mouse groups to which the Cannabis sativa extract (CE) (OT1) and extract mixtures (OT2 to OT6) of the present disclosure were administered, respectively, showed a significant decrease in weight gain compared to the mouse groups to which DMSO and phloretin were administered, respectively.

This suggests that administration of each of the Cannabis sativa extract (CE) (OT1) and extract mixtures (OT2 to OT6) of the present disclosure has the effect of suppressing weight gain. It was confirmed that this effect of suppressing weight gain was not an effect attributable to a difference in food intake, from the fact that there was no difference in food intake between the test groups.

### Test Example 5: Palatability Test

Tea beverages were prepared by diluting each of the Cannabis sativa extract (CE) (OT1) and extract mixtures (OT2 to OT6) of the present disclosure. Each of the tea beverages were tasted by 10 panelists, and the taste and flavor thereof were scored on a 10-point scale (1 to 10). The average values of the scores (any fraction of 0.5 or more is rounded up to the next higher whole number) are shown in Table 3 below. In the scores in Table 3 below, a higher score indicates higher palatability.

TABLE 3

| | OT1 | OT2 | OT3 | OT4 | OT5 | OT6 |
|---|---|---|---|---|---|---|
| Taste | 6.0 | 6.0 | 6.5 | 7.0 | 7.5 | 6.0 |
| Flavor | 6.0 | 6.5 | 6.5 | 7.0 | 7.5 | 7.0 |
| Overall palatability | 6.0 | 6.0 | 7.0 | 7.0 | 7.5 | 6.5 |
| (average) | | | | | | |

(unit: score)

Referring to Table 3 above, it can be seen that, in the case of OT1 composed of the Cannabis sativa extract (CE) alone, the palatability was lowered due to the unique taste and flavor of the Cannabis sativa extract, and in the case of the mixtures OT2 to OT6, the palatability increased while the unique taste and flavor of the Cannabis sativa extract were neutralized by the other extracts.

In particular, it was confirmed that, in the case of OT3 to OT5, the effect of preventing and treating obesity was excellent, and the palatability greatly increased while the taste and flavor were highly evaluated.

Therefore, each of the extract mixtures OT3 to OT5 according to the present disclosure may provide a functional food having an excellent effect on the prevention and treatment of obesity while having higher flavor and taste palatability.

As described above, the present disclosure may provide a composition for preventing and treating obesity, which contains an extract of the natural product Cannabis sativa as an active ingredient, and thus has little or no side effects when taken or administered, and may suppress the body weight and body fat gain caused by a high-fat diet in a diet-induced obesity mouse model.

Although the preferred embodiments of the present disclosure have been described in detail above, the scope of the present disclosure is not limited thereto, and various modified and improved forms made by those skilled in the art on the basis of the basic concept of the present disclosure defined in the appended claims also fall within the scope of the present disclosure.

What is claimed is:

1. A composition for preventing and treating obesity containing a Cannabis sativa extract as an active ingredient.

2. The composition of claim 1, wherein the Cannabis sativa extract contains cannabidiol and terpene.

3. The composition of claim 1, wherein the composition suppresses obesity by inhibiting adipocyte differentiation-stimulating factors.

4. The composition of claim 3, wherein the adipocyte differentiation-stimulating factors include PPARγ (peroxisome proliferator-activated receptor γ), C/EBPα (CCAAT/enhancer-binding protein α), and fatty acid synthase.

5. The composition of claim 1, wherein the Cannabis sativa extract is obtained by extraction with an extraction solvent selected from the group consisting of water, a C₁ to C₆ lower alcohol, and a mixture thereof.

6. A food composition for preventing obesity comprising the composition according to claim 1.

7. A pharmaceutical composition for treating obesity comprising the composition according to claim 1.
